(19) United States
(12) Patent Application Publication
(10) Pub. No.: US 2003/0221234 A1
(43) Pub. Date: Dec. 4, 2003

(54) Visor structure with an elastic absorbent soft band in a bevel mode

(76) Inventor: Tai-Kuang Wang, Taipei (TW)

Correspondence Address: TROXELL LAW OFFICE PLLC, 5205 LEESBURG PIKE, SUITE 1404, FALLS CHURCH, VA, 22041, US

(21) Appl. No.: 10/155,128

(22) Filed: May 28, 2002

(51) Int. Cl. A61F 9/00

(52) U.S. Cl. 2/12; 2/181

(57) ABSTRACT

A visor structure with an elastic band connecting in a bevel mode, it comprises a peak, a front panel and a band; the band is made of elastic fabric (such as elastic terry or elastic fleece); the front panel and the band are both cut in a distinctive bevel mode to connect with each other to thereby form the visor with a perfect streamline. When the visor is worn on, the band is pulled out and its elastic fabric can be stretched out horizontally to suit the different head sizes of the wearer, to make the band look quite even, tidy and not being crimpled to influence the entire appearance of the visor structure. The visor provides comfort no matter it is worn normally or in a reverse way by virtue that the ears of the wearer always contact the soft band.

### BACKGROUND OF THE INVENTION

1. Field of the Invention

The present invention is related to a visor structure, and especially to a visor structure with an elastic absorbent soft band connecting in a bevel mode that provides comfort for a user no matter the visor is worn normally or in a reverse way.

2. Description of the Prior Art

Fashions and kinds of headgears are variant, in addition to the functions of sun shading and keeping warm, they can provide protection in exercise. Headgears normally seen are caps, that is, the crown of a headgear is comprised of a plurality of panels, hence it is unfit for ventilation and perspiration discharge, and thereby is not good for use in summer season and in exercise.

There have been visors improved on the defect mentioned above, they can be adjusted by providing a rear opening “A” with a closure portion “B”, as shown in FIG. 1. Closure “B” generally is made of plastic and integrally shaped with a peak “C”; and when a user wears the visor, the visor will contact and abrade the ears of the wearer resulting a bad sense of tactility; and if closure “B” is adjusted to expose too much excessive portion it'll detract from esthetic appearance, thereby, such visor is not desired.

The plastic visor is just one among various types of visor, most visors available presently are made of rigid fabric with portions of peak, front panel and band in which front panel is connected with band orthogonally, as shown in FIGS. 1a and 1b, though rigid fabric has better sense of touch than that of plastic, it is still too stiff, in view of this, when the ears of the wearer contact the ends of front panel of visor the wearer still feel discomfort, and thereby they are not desired.

Therefore, it is the motive of the present invention to improve the structure of the conventional visors with the above defects by providing a visor structure with an elastic absorbent soft band connecting in a bevel mode that provides comfort wearing, and does not expose an excessive portion to detract from esthetic appearance.

### SUMMARY OF THE INVENTION

The primary object of the present invention is to provide a visor structure with an elastic absorbent soft band connecting in a bevel mode, which has the features of perspiration absorbency, soft touch, and, the edge of peak and the end of front panel aligned in a perfect streamline.

The secondary object of the present invention is to provide a visor structure with an elastic absorbent soft band connecting in a bevel mode; which can automatically make adjustment of the size itself in pursuance of the size of wearer's head, it does not expose an excessive portion to detract from esthetic appearance.

To get the above stated objects, the present invention is comprised of a peak, a front panel and a band, wherein, the band is made of elastic fabric (such as elastic terry or elastic fleece); the band and the front panel are both cut in a distinctive bevel mode to connect with each other to thereby form the edge of peak and the connecting line of front panel and band a perfect streamline, furthermore the bevel mode makes the ears of the wearer always contact the soft band no matter the visor is worn normally or in a reverse way. Besides, the band has a good feature of perspiration absorbency, it does not need an additional sweatband, which is of cost saving.

The present invention will be apparent in its detailed structure and characteristics after reading the detailed description of the preferred embodiment thereof in reference to the accompanying drawings.

### BRIEF DESCRIPTION OF THE DRAWINGS

FIG. 1a is a perspective view showing the appearance of a conventional visor;

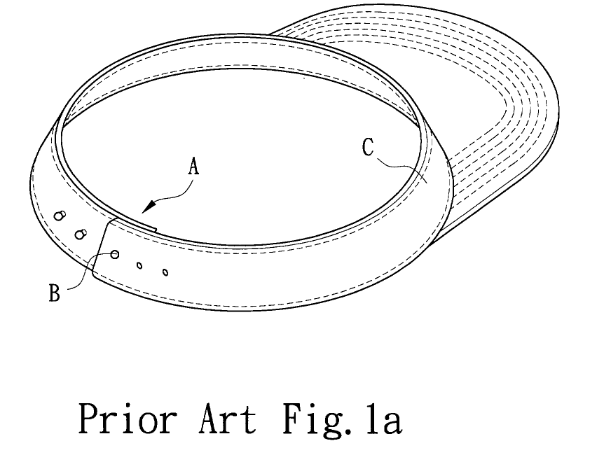

FIG. 1b is a perspective view showing the appearance of another conventional visor;

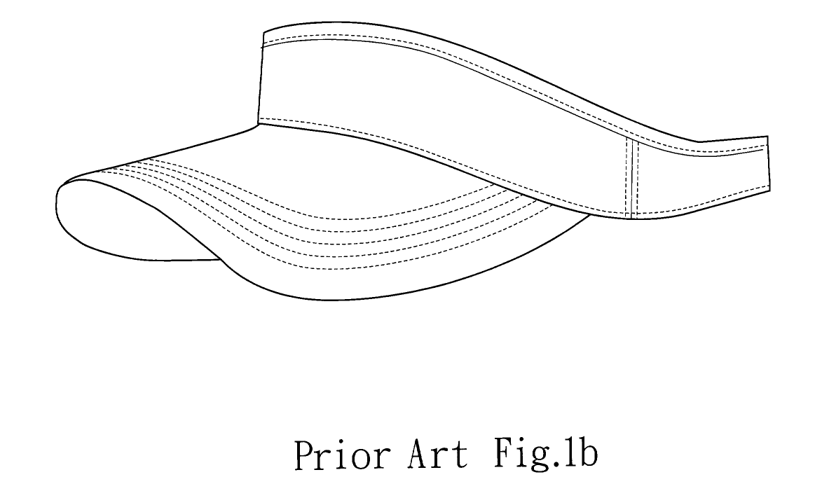

FIG. 2 is a perspective view showing the appearance of an embodiment of the present invention;

FIG. 3 is an analytic perspective view of the embodiment of the present invention;

FIG. 4 is a schematic view showing use of the embodiment of the present invention;

FIG. 5 is a schematic view showing the embodiment of the present invention being used in a reverse way.

### DETAILED DESCRIPTION OF THE PREFERRED EMBODIMENT

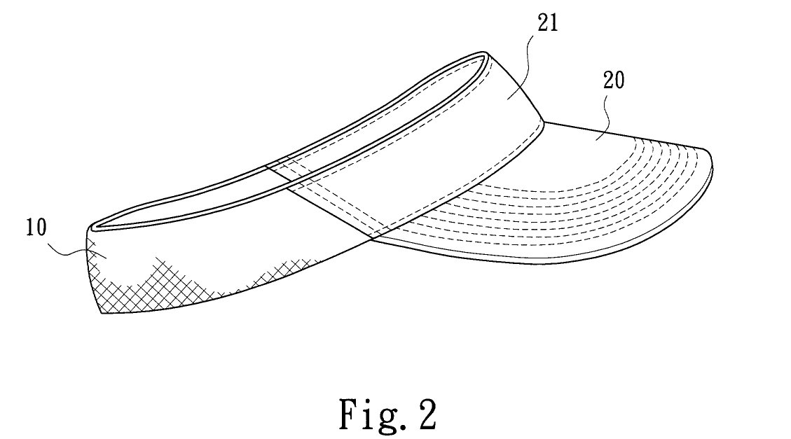

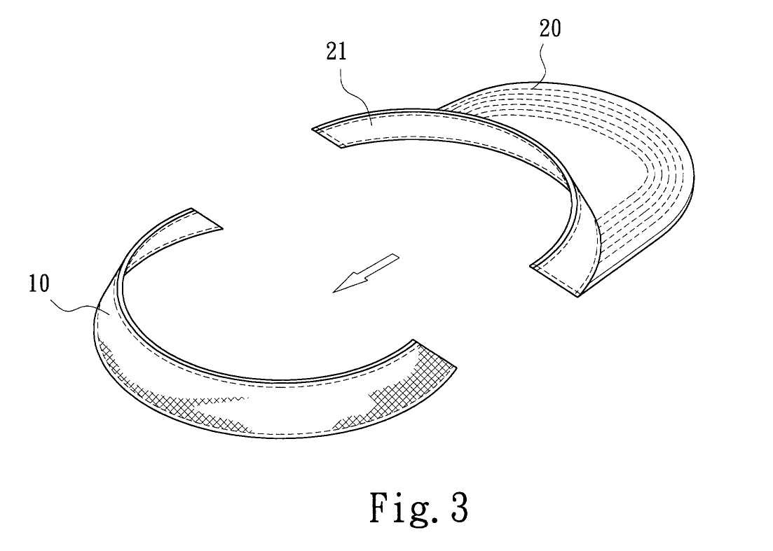

Referring to FIGS. 2 and 3, the visor of the present invention is comprised of a band 10, a front panel 21 and a peak 20 sewn together, wherein, the band 10 is made of elastic fabric such as elastic terry or fleece with a suitable width, two pairs of connecting end of the band 10 and front panel are cut both in beveled way; the peak 20 is curved and used to shade sunshine, the front panel 21 is extended upwardly to form a connecting portion which forms an angle with the peak 20, the bottom of the front panel 21 is sewn with the upper side of the peak 20. Two ends of the front panel 21 each forms a distinctively beveled connecting end in corresponding to the two beveled connecting ends of the band 10, thereby, the two beveled connecting ends of the band 10 are butt-connected with the beveled connecting ends of the front panel 21 to form the assembly of the visor structure with a perfect streamline.

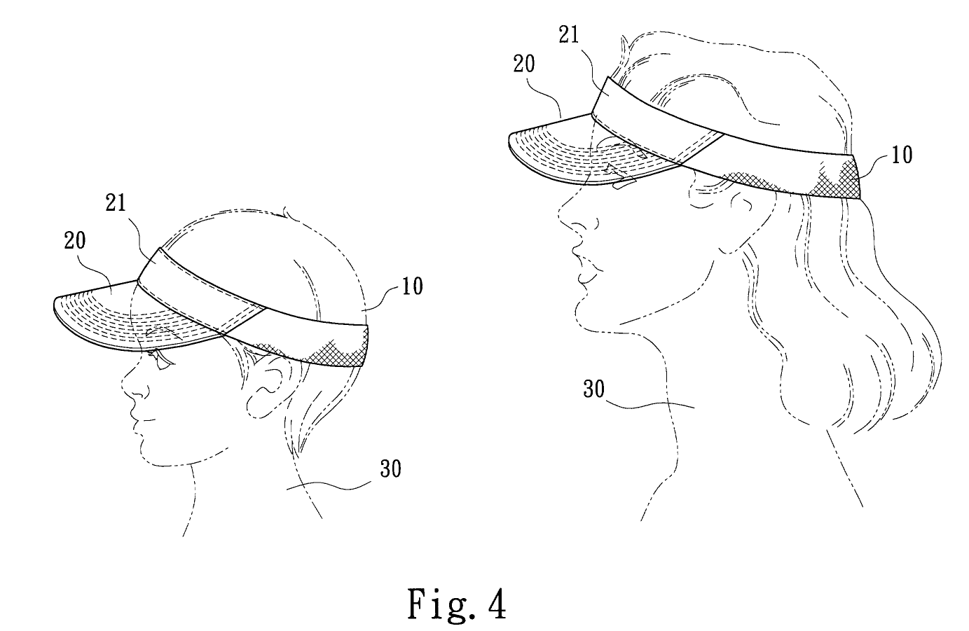

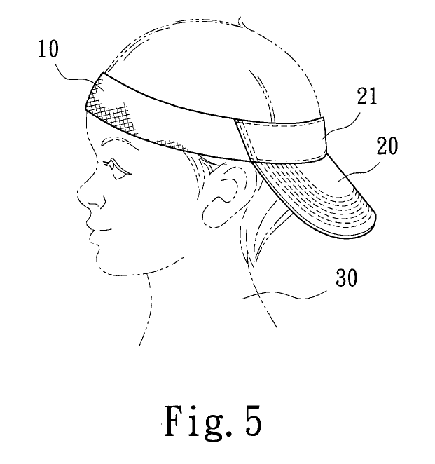

Referring to FIG. 4, if a wearer 30 wearing the visor of the present invention is a child or one with a smaller head size, he can wear the visor of the present invention directly and conveniently; when an adult or one with a larger head size wears the visor of the present invention, he can wear the visor in the same way on his head. By virtue that the head size of the wearer is larger than the normal size of the visor, when in wearing, the band 10 made of elastic fabric has excellent flexibility and can thereby be stretched to make the size of the visor larger than that of before being stretched, and then the wearer with a larger head size can wear the visor on. And by virtue that the band 10 is made of elastic fabric, although it is same with the conventional ones in the way of wearing and in contacting and abrading the ears during wearing, its soft character is better than that of the rigid fabric or plastic band, thereby, the visor provides comfort no matter the visor is worn normally or in a reverse way (such as shown in FIG. 5) by virtue that the ears of the wearer always contact the soft band 10; hence, a best effect of covering can be obtained to make the wearer cozy. And more, when the visor of the present invention is worn on a wearer with a larger head size, by the fact that the band 10 is directly stretched out horizontally, the band 10 looks quite even, tidy and will not be crimpled to influence the entire appearance of the visor structure.

The visor structure of the present invention thereby has the following advantages:

1. The band in the present invention is made of elastic fabric with good features of ventilation and perspiration absorbency, it does not need an additional sweatband to absorb sweat; this not only can reduce cost, but also can increase the practicality of the present invention.

2. The present invention has an adjustable band to allow direct wearing in a normal way, this can omit the cumbersome action of adjustment of the size of the band as is done for a conventional visor.

3. The size of visor of the present invention adjusted by elastic fabric of the band; thereby, the band will not crimple and affect the appearance of the entire visor.

In conclusion, the present invention not only can get rid of the defect of conventional visors needing the cumbersome action of adjustment of the size of the band and having inferior sense of touch, but also provides the band made of elastic fabric in favor of adjustment of the size of the band to suit the different head sizes of wearer; it is doubtlessly practicable. Having thus described my invention, what I claim as new and desire to be secured by Letters Patent of the United States are:

1. A visor structure with an elastic absorbent soft band connecting in a bevel mode comprising:
a peak which is curved and used to shade sunshine, a front panel which is extended upwardly to form a connecting portion which forms an angle with the peak, wherein said front panel and a band with two connecting ends cut both in beveled way, thereby, the edge of peak and the connecting line of front panel and band align in a perfect streamline when said band and said front panel are combined with each other.

2. The visor structure as in claim 1, wherein, said band is made of elastic fabric such as elastic terry or fleece, by virtue of perspiration absorbency of the band itself, thereby there is no need of an additional sweatband.

3. The visor structure as in claim 2, wherein, said band is made of elastic fabric with features of Wick-Dry, which wicks moisture at the inner layer and evaporates at the outer layer for superior dry.

4. The visor structure as in claim 1, wherein, two connecting ends of a front panel form distinctively beveled connecting ends in corresponding to two beveled connecting ends of said band.
